(19) United States
(12) Patent Application Publication
(10) Pub. No.: US 2021/0029907 A1
(43) Pub. Date: Feb. 4, 2021

(54) STEVIA CULTIVARS A01, A03, A05, A06, A07, AND A08

(71) Applicant: SWEET GREEN FIELDS USA LLC, BELLINGHAM, WA (US)

(72) Inventor: Mel Clinton JACKSON, Waikoloa, HI (US)

(21) Appl. No.: 16/994,142

(22) Filed: Aug. 14, 2020

Related U.S. Application Data
(63) Continuation of application No. 16/358,436, filed on Mar. 19, 2019, now abandoned.
(60) Provisional application No. 62/647,036, filed on Mar. 23, 2018.

(51) Int. Cl. A01H 6/14; A01H 5/10

(52) U.S. Cl. CPC A01H 6/1488; A01H 5/10

(57) ABSTRACT

Stevia cultivars, designated A01, A03, A05, A06, A07 and A08, are disclosed. Plant parts of stevia cultivars, the plants of stevia, methods for producing a stevia plant produced by crossing the cultivars with itself or another stevia variety, hybrid stevia seeds and plants produced by crossing the cultivar with another stevia cultivar and plant products e.g., glycosides, are within the scope of the invention.

This application is a continuation-in-part of U.S. application Ser. No. 16/358,436, filed Mar. 19, 2019, which claims priority to U.S. Provisional Application Ser. No. 62/647,036, filed Mar. 23, 2018. The entirety of the aforementioned application is incorporated herein by reference.

### FIELD

The present disclosure relates to a genus of new stevia cultivars and methods for genotyping these stevia cultivars and genetically distinguishing these cultivars from each other and other publicly known stevia varieties.

### BACKGROUND

The present disclosure relates to stevia (Stevia rebaudiana) cultivars designated A01, A03, A05, A06, A07, and A08, including seeds, plants, and hybrids. This disclosure further relates to a method for producing stevia seed and plants from A01, A03, A05, A06, A07, and A08 cultivars.

Stevia is an important and valuable field crop for the production of sweeteners, sugar substitutes, and other consumable ingredients. A goal of stevia plant breeders is to develop stable, high yielding stevia cultivars of stevia species that are commercially useful. This means cultivars that have suitable amounts and quality of sweeteners, sugar substitutes, and other consumable ingredients.

The development of new stevia cultivars requires the evaluation and selection of parental plants and crossing of these parents to produce improved cultivars

### SUMMARY

One aspect of the present application relates to a stevia plant that comprises the allele combination at genetic marker sites listed below: A/A or A/B at genetic marker site GANE01021955; A/A or A/B at genetic marker site GANE01016707; A/A or A/B at genetic marker site GANE01004406; A/B or B/B at genetic marker site WOUH023266.1; A/A at genetic marker site GANE01025251; A/A or B/B at genetic marker site UDP85C2; and A/A at genetic marker site WOUH01005557.1, with the proviso that when GANE01021955 is A/B, GANE01004406 is A/B.

In some embodiments, the stevia plant further comprising one or more of the alleles selected from the group consisting of: A/B or B/B at genetic marker site GANE01000877; A/A or A/B at genetic marker site GANE01003004; A/A or B/B at genetic marker site GANE01011595; A/B or B/B at genetic marker site GANE01015568; A/B or B/B at genetic marker site WOUH023266.1.2; A/B or B/B at genetic marker site UDP74G1; and A/A or B/B at genetic marker site GANE01016432.

Another aspect or the present application relates to a plant, or a plant part thereof, produced by growing the plant of the present application.

Another aspect or the present application relates to an extract of the stevia plant of the present application, wherein the extract comprises one or more steviol glycosides.

Another aspect or the present application relates to a tissue or cell culture of regenerable cells produced from the plant of the present application.

Another aspect or the present application relates to a method of producing stevia seeds, comprising: (a) planting seeds of the stevia plant of the present application; (b) cultivating stevia plants resulting from the seeds until the plants bear flowers; (c) allowing fertilization of the flowers of the plants; and (d) harvesting seeds produced from the plants.

Another aspect or the present application relates to a method of vegetatively propagating a stevia plant, the method comprising: (a) collecting tissue or cells capable of being propagated from the plant according to claim 1; (b) cultivating the tissue or cells of (a) to obtain proliferated shoots; and (c) rooting said the shoots to obtain rooted plantlets or cultivating said tissue or cells to obtain proliferated shoots, or plantlets.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIGS. 1A and 1B are photographs of the A01 stevia plant; FIG. 1A is a photograph of single leaves; FIG. 1B is a photograph of a growing plant.

FIGS. 2A and 2B are photographs of the A03 stevia plant; FIG. 2A is a photograph of single leaves; FIG. 2B is a photograph of a growing plants.

FIGS. 3A and 3B are photographs of the A05 stevia plant; FIG. 3A is a photograph of single leaves; FIG. 3B is a photograph of plants with colored sections.

FIGS. 4A and 4B are photographs of the A06 stevia plant; FIG. 4A is a photograph of single leaves; FIG. 4B is a photograph of growing plants.

FIGS. 5A and 5B are photographs of the A07 stevia plant; FIG. 5A is a photograph of single leaves; FIG. 5B is a photograph of growing plants.

FIGS. 6A and 6B are photographs of the A08 stevia plant; FIG. 6A is a photograph of single leaves; FIG. 6B is a photograph of growing plants.

FIG. 7 depicts a multidimensional scaling analysis based on identity by state (IBS) showing the differentiation in genetic space.

FIGS. 8A to 8P depict genotyping S. rebaudiana lines. In particular, each figure is a 2% agarose gel photograph of S. rebaudiana PCR amplification products (or extensions) using the primers described in Table 7, optionally in combination with restriction enzyme digestion as indicated. Each set of primers was used to amplify a DNA fragment in the form of a specific marker product for genotyping alleles at an informative locus. Each figure presents an analysis of a plurality of S. rebaudiana lines with respect to the specific marker (as indicated).

FIG. 8A is shows amplified CAPS marker products at the GANE01021955 locus digested with AvaI. The first lane represents a ladder showing fragment size. Lane 2 represents a positive control of an undigested PCR product. Lane 3 is cultivar A01 showing a homozygous missing restriction site. Lane 4 is cultivar A03 showing a heterozygous restriction site. Lane 5 is cultivar A05 showing a heterozygous restriction site. Lane 6 is cultivar A06 showing a heterozygous restriction site. Lane 7 is cultivar A07 showing a homozygous missing restriction site. Lane 8 is cultivar A08 showing a homozygous missing restriction site. Lane 9 is control cultivar A4 showing a homozygous restriction site. Lanes 10 is control cultivar A10 showing missing data. Lane 11 is control cultivar A13 showing a homozygous missing restriction site. Lane 12 is control cultivar #1 showing a heterozygous restriction site. Lane 13 is control cultivar #2 showing a homozygous restriction site. Lane 14 is control cultivar M7 showing a heterozygous restriction site.

FIG. 8B shows amplified CAPS marker products at the GANE01016707 locus digested with DraI. The first lane represents a ladder showing fragment size. Lane 2 represents a positive control of an undigested PCR product. Lane 3 is cultivar A01 showing a heterozygous restriction site. Lane 4 is cultivar A03 showing a homozygous absence of a restriction site. Lane 5 is cultivar A05 showing a homozygous absence of a restriction site. Lane 6 is cultivar A06 showing a homozygous absence of a restriction site. Lane 7 is cultivar A07 showing a heterozygous restriction site. Lane 8 is cultivar A08 showing a heterozygous restriction site. Lane 9 is a ladder showing fragment size. Lane 10 is a positive control. Lane 11 is control cultivar #1 showing a heterozygous restriction site. Lanes 12 is control cultivar #2 showing homozygous absence of a restriction site. Lane 13 is control cultivar A4 showing a heterzygous restriction site. Lane 14 is control cultivar A10 showing a heterozygous restriction site. Lane 15 is control cultivar AH showing a homozygous restriction site. Lane 16 is control cultivar M7 showing a heterozygous restriction site.

FIG. 8C shows amplified PCR marker products at the GANE01016455 locus. The first lane represents a ladder showing fragment size. Lane 2 represents a negative control of no DNA. Lane 3 is cultivar A1 showing a heterozygous reaction. Lane 4 is cultivar A3 showing a heterozygous reaction. Lane 5 is cultivar A5 showing a heterozygous reaction. Lane 6 is cultivar A6 showing a heterozygous reaction. Lane 7 is cultivar A7 showing a homozygous band for allele B. Lane 8 is cultivar A8 showing a homozygous band for allele A. Lane 9 is a negative control. Lane 10 is another ladder Lane 12 is a ladder showing fragment size. Lane 13 is a negative control. Lane 14 is control cultivar #1 showing a homozygous band for allele B. Lane 15 is control cultivar #2 showing homozygous band for allele A. Lane 16 is cultivar A4 showing a heterzygous reaction. Lane 17 is control cultivar A10 showing a homozygous band for allele A. Lane 18 is control cultivar AH showing a heterozygous reaction. Lane 19 is control cultivar M7 showing a band for allele B.

FIG. 8D shows amplified CAPS marker products at the GANE01004406 locus digested with HphI. (Top panel) Lane 1 on the top is a ladder. Lane 2 is empty. Lane 3 is cultivar A03 which is heterozygous for the cut site. Lane 4 is A05 which is heterozygous for the cut site. Lane 5 is cultivar A6 which is heterozygous for the cut site. Lane 6 is A7 which is heterozygous for the cut site. Lane 7 is cultivar A08 which is homozygous for no cut. Lane 8 is a negative control. Lane 9 is an undigested positive control. Lane 10 is a ladder. Lane 11 is a ladder. Lane 12 is cultivar A01 which is homozygous for no restriction site. (Bottom panel) Lane 1 is a ladder, Lane 2 is a positive control, Lane 3 is control cultivar #1 and is homozygous no restriction site, Lane 4 is control cultivar #2 and heterozygous for the restriction site. Lane 5 is cultivar A05 and is homozygous for no restriction site. Lane 6 is control cultivar A10 which is heterozygous for the restriction site. Lane 7 is control cultivar AH and it is homozygous for no restriction site. Lane 8 is control cultivar M7 is homozygous for no restriction site.

FIG. 8E shows amplified CAPS marker products at the GANE01025251 locus digested with AluI. Lane 1 is a ladder. Lane 2 is a positive control. Lane 3 is cultivar A01 and is homozygous for no restriction site. Lane 4 is cultivar A03 which is heterozygous for the restriction site. Lane 5 is cultivar A05 which is heterozygous for the restriction site. Lane 6 is cultivar A06 which is heterozygous for the restriction site. Lane 7 is cultivar A07 which is heterozygous for the restriction site. Lane 8 is cultivar A08 which is homozygous for no restriction site. Lane 9 is a negative control of water. Lane 10 is a size ladder. Lane 11 is a size ladder. Lane 12 is a positive control. Lane 13 is control cultivar #1 which is homozygous for no restriction site. Lane 14 is control cultivar #2 which is heterozygous for the restriction site. Lane 15 is control cultivar A4 which is heterozygous for the restriction site. Lane 16 is control cultivar A10 which is homozygous for no restriction site. Lane 17 is control cultivar AH which is homozygous for no cut site. Lane 18 is control cultivar M7 which is homozygous for no restriction site.

FIG. 8F shows amplified CAPS marker products at the GANE01000877 locus digested with HphI. Lane 1 is a ladder. Lane 2 is a positive control. Lane 3 is cultivar A01 which is heterozygous for the cut site. Lane 4 is cultivar A03 which is homozygous for the cut site. Lane 5 is cultivar A05 which is homozygous for the cut site. Lane 6 is cultivar A06 which is homozygous for the cut site. Lane 7 is cultivar A07 which is homozygous for the cut site. Lane 8 is cultivar A08 which is which is homozygous for the cut site. Lane 9 is a ladder. Lane 10 is a positive control. Lane 11 is control cultivar #1 which is homozygous for the cut site. Lane 12 is control cultivar #2 which is homozygous for the cut site. Lane 13 which is control cultivar A4 which is homozygous for the cut site. Lane 14 is control cultivar A10 which is homozygous for the cut site. Lane 15 is control cultivar AH which is homozygous for the cut site. Lane 16 is control cultivar M7 which is homozygous for the cut site.

FIG. 8G shows amplified CAPS marker products at the GANE01003004 locus digested with Hha. (Top panel) Lane 1 is the size ladder. Lane 2 is positive control. Lane 3 is cultivar A01 which is homozygous for no cut. Lane 4 is cultivar A03 which is heterozygous for the cut site. Lane 5 is cultivar A05 which is heterozygous for the cut site. Land 6 is cultivar A06 which is heterozygous for the cut site. Lane A7 is cultivar A07 which is heterozygous for the cut site. Lane 8 is cultivar A08 which is homozygous for no cut. Lane 9 is a negative control. Lane 10 is a ladder. (Bottom panel) Lane 1 is a size ladder. Lane 2 is a positive control. Lane 3 is control cultivar #1 which is homozygous not cut. Lane 4 is control cultivar #2 which is missing data. Lane 5 is control cultivar A4 which is heterozygous for the cut site. Lane 6 is control cultivar A10 which is heterozygous for the cut site. Lane 7 is control cultivar AH which is homozygous for no cut. Lane 8 is control cultivar M7 which is heterozygous for the cut site.

FIG. 8H shows amplified CAPS marker products at the GANE01011595 locus digested with EcoP15I. (Top panel) Lane 1 is a ladder. Lane 2 is a positive control. Lane 3 is cultivar A01 which is heterozygous for the cut site. Lane 4 is cultivar A03 which is heterozygous for the cut site. Lane 5 is cultivar A05 which is homozygous for the cut site. Lane 6 is cultivar A06 which is homozygous for the cut site. Lane 7 is cultivar A07 which is heterozygous for the cut site. Lane 8 is cultivar A08 which is heterozygous for the cut site. (Bottom panel) Lane 1 is a ladder. Lane 2 is a positive control. Lane 3 is control cultivar #1 which homozygous for no cut site. Lane 4 is control cultivar #2 which is missing data. Lane 5 is control cultivar A4 which is homozygous for no cut. Lane 6 is control cultivar A10 which is heterozygous for the cut site. Lane 7 is control cultivar AH which is heterozygous for the cut site. Lane 8 is control cultivar M7 which is homozygous for the cut site.

FIG. 8I shows amplified CAPS marker products at the GANE01015568 locus digested with BamHI and BstYI. (Top panel) Lane 1 is a size ladder. Lane 2 is a positive control. Lane 3 is second positive control. Lane 4 is cultivar A01 which is heterozygous for the cut site. Lane 5 is cultivar A03 which is heterozygous for the cut site. Lane 6 is cultivar A05 which is which is heterozygous for the cut site. Lane 7 is cultivar A06 which is heterozygous for the cut site. Lane 8 is cultivar A07 which is heterozygous for the cut site. Lane 9 is cultivar A08 which is homozygous for the cut site. (Bottom panel) Lane 1 is a size ladder. Lane 2 is a positive control. Lane 3 is second positive control. Lane 4 is control cultivar #1 which is homozygous for the cut site. Lane 5 is control cultivar #2 which is missing data. Lane 6 is control cultivar A4 which is heterozygous for the cut site. Lane 7 is control cultivar A10 which is heterozygous for the cut site. Lane 8 is cultivar control cultivar AH which is heterozygous for the cut site. Lane 9 is control cultivar M7 which is homozygous for the cut site.

FIG. 8J shows amplified PCR marker products at the GANE01016432 locus. (Top panel) Lane 1 is a ladder. Lane 2 is cultivar A01 which shows the band for allele B. Lane 3 is cultivar A03 which has the band for allele A. Lane 4 is cultivar A05 which has the band for allele A. Lane 5 is cultivar A06 which has the band for allele B. Lane 6 is the cultivar A07 which has the band for allele A. Lane 8 is the cultivar A08 which has the band for allele B. (Bottom panel) Lane 1 is a ladder. Lane 2 is control cultivar #1 which has allele A. Lane 3 is control cultivar #2 which has allele A. Lane 4 is control cultivar A4 which is allele A. Lane 5 is control cultivar A10 which has allele A. Lane 6 is control cultivar AH which is allele B. Lane 7 is control cultivar M7 which is allele B.

FIG. 8K shows amplified CAPS marker products at the WOUH023266.1 locus digested with Aval. (Top panel) Lane 1 is a size ladder. Lane 2 is a positive control. Lane 3 is cultivar A01 which shows a heterozygous restriction site. Lane 4 is cultivar A03 which shows a heterozygous restriction site. Lane 5 is cultivar A05 which shows a heterozygous restriction site. Lane 6 is cultivar A06 which shows a heterozygous restriction site. Lane 7 is cultivar A07 which is homozygous for the restriction site. Lane 8 is cultivar A08 which is homozygous for the cut site. (Bottom panel) Lane 1 is a ladder. Lane 2 is a positive control. Lane 3 is control cultivar #1 which shows a heterozygous restriction site. Lane 4 is control cultivar #2 which is homozygous for no cut site. Lane 5 is control cultivar A4 which is homozygous for the cut site. Lane 6 is control cultivar A10 which is homozygous for the cut site. Lane 7 is control cultivar AH which shows a heterozygous restriction site. Lane 8 is the control cultivar M7 which shows a heterozygous restriction site.

FIG. 8L shows amplified CAPS marker products at the WOUH023266.1.2 locus digested with HphI. Lane 1 is ladder. Lane 2 is a positive control. Lane 3 is cultivar A01 which is homozygous for the cut. Lane 4 is cultivar A03 which shows a heterozygous restriction site. Lane 5 is cultivar A05 which shows a heterozygous restriction site. Lane 6 is cultivar A06 which shows a heterozygous restriction site. Lane 7 is cultivar A07 which is homozygous for the cut site. Lane 8 is cultivar A08 which is homozygous for the cut site. Lane 9 is control cultivar #1 which is homozygous for the cut cite. Lane 10 is control cultivar #2 which is homozygous no cut site. Lane 11 is control cultivar A4 which is homozygous for the cut site. Lane 12 is control cultivar A10 which is homozygous for the cut site. Lane 13 is control cultivar AH which shows a heterozygous restriction site. Lane 14 is control cultivar M7 which shows a heterozygous restriction site.

FIG. 8M shows amplified CAPS marker products at the WOUH01005557.1 locus digested with HhaI. Lane 1 is a size ladder. Lane 2 is positive control. Lane 3 is cultivar A01 which is homozygous for no cut. Lane 4 is cultivar A03 which is homozygous for no cut. Lane 5 is cultivar A05 which is homozygous for no cut. Lane 6 is cultivar A06 which is homozygous for no cut. Lane 7 is cultivar A07 which is homozygous for no cut. Lane 8 is cultivar A08 which is homozygous for no cut. Lane 9 is ladder. Lane 10 is positive control. Lane 11 is control cultivar #1 is homozygous for the cut. Lane 12 is control cultivar #2 which is missing data. Lane 13 is control cultivar A4 which is homozygous for no cut. Lane 14 is control cultivar A10 which is homozygous for no cut. Lane 15 is control cultivar AH which is heterozygous for the cut site. Line 16 is control cultivar M7 which is homozygous for no cut.

FIG. 8N shows amplified CAPS marker products at the UDP74G1 locus digested with AluI. Lane 1 is a ladder. Lane 2 is a positive control. Lane 3 is cultivar A01 which has multiple cut sites. Lane 4 is cultivar A03 which has multiple cut sites. Lane 5 is cultivar A05 which has multiple cut sites. Lane 6 is cultivar A06 which has one cut site. Lane 7 is cultivar A07 which has multiple cut sites. Lane 8 is cultivar A08 which has multiple cut sites. Lane 9 is a ladder. Lane 10 is positive control. Lane 11 is control cultivar #1 which has multiple cut sites. Lane 12 is control cultivar #2 which has on cut site. Lane 13 is control cultivar A4 which has multiple cut sites. Lane 14 is control cultivar A10 which has one cut site. Lane 15 is control cultivar AH which has one cut site. Lane 16 is control cultivar M7 has multiple cut sites.

FIG. 8O shows amplified ISSR marker products at the DVE44 locus. Lane 1 is a ladder. Lane 2 is cultivar A01 and has 3 major bands. Lane 3 is cultivar A03 which has 4 major bands. Lane 4 is cultivar A05 which has 5 major bands. Lane 5 is cultivar A06 which has 5 major bands. Lane 6 is cultivar A07 which has 4 major bands. Lane 7 is cultivar A08 which has 4 major bands. Lane 8 is control cultivar #1 which has 3 major bands. Lane 9 is control cultivar #2 which has 3 major bands. Lane 10 is control cultivar A4 which has 4 major bands. Lane 11 is control cultivar A10 which has 6 major bands. Lane 12 is control cultivar AH which has 5 major bands. Lane 13 is control cultivar M7 which has 2 major bands. Lane 14 is a negative control.

FIG. 8P shows amplified PCR marker products at the UDP85C2 locus. Lane 1 is a ladder. Lane 2 is cultivar A01 which shows a band at allele A. Lane 3 is cultivar A03 which shows allele A. Lane 4 is cultivar A05 which shows allele B. Lane 5 is cultivar A06 which shows allele B. Lane 6 is cultivar A07 which shows allele B. Lane 7 is cultivar A08 which shows allele B. Lane 8 is control cultivar #1 which shows allele A. Lane 9 is control cultivar 2 which shows allele A. Lane 10 is control cultivar A4 which shows allele A. Lane 11 is control cultivar A10 which shows #allele B. Lane 12 is control cultivar AH which shows allele B. Lane 13 shows control cultivar M& which shows allele A. Lane 14 is a negative control of water.

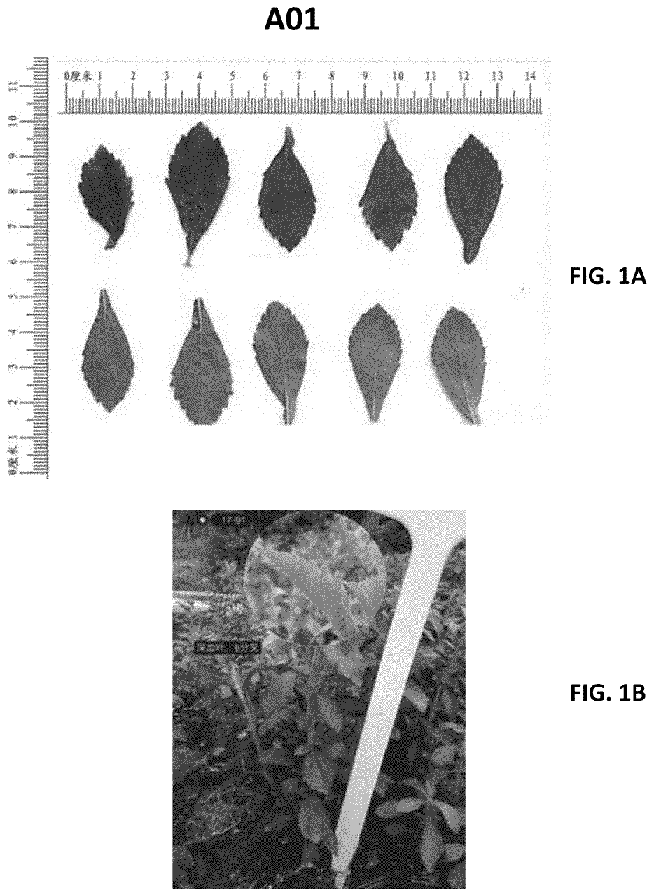

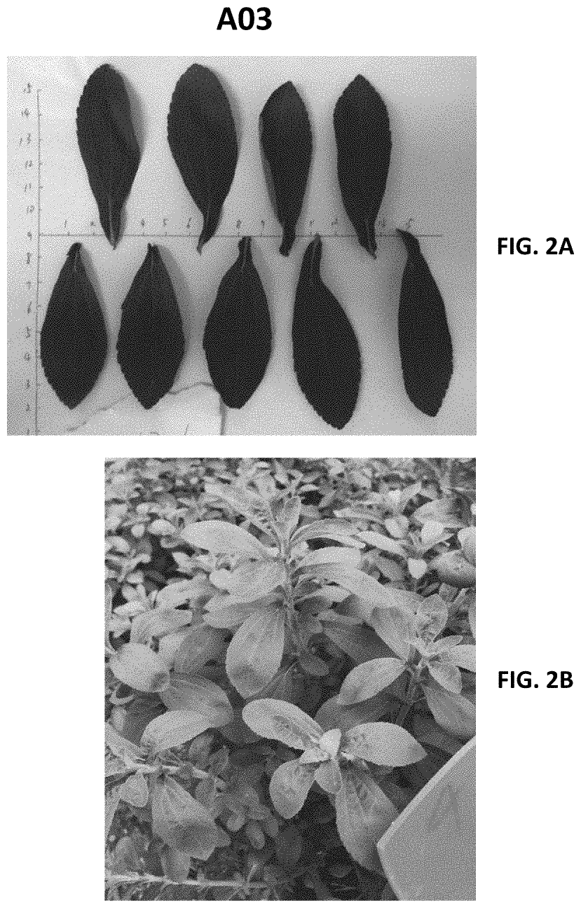

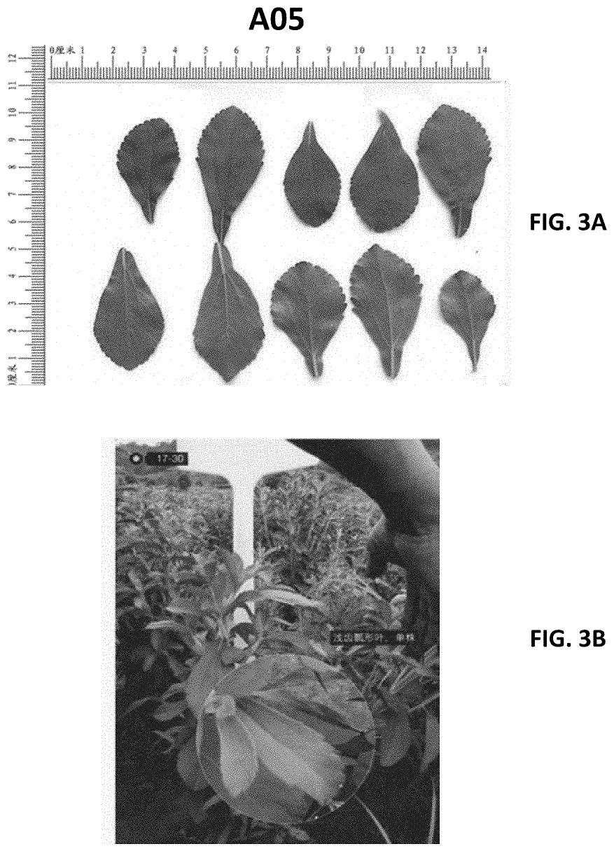

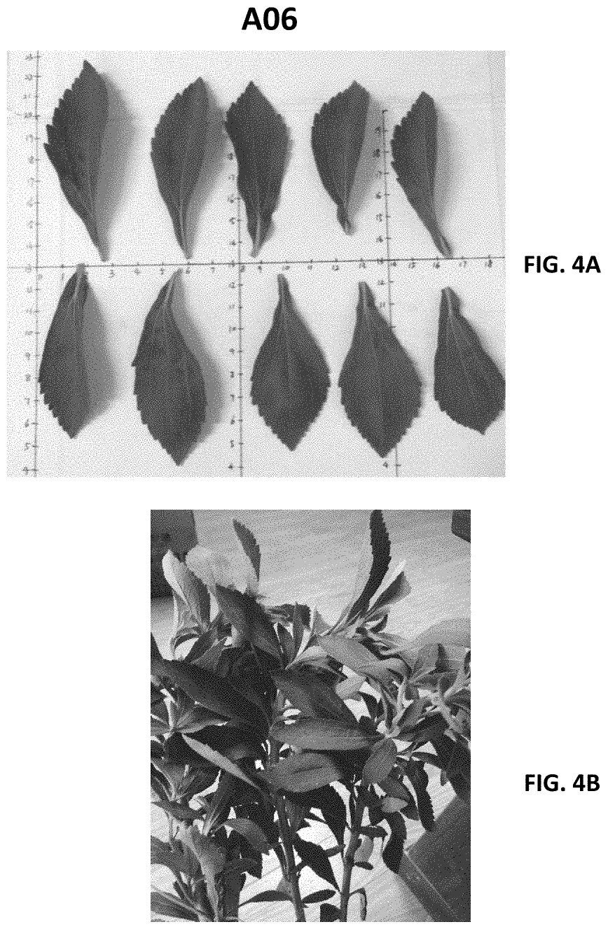

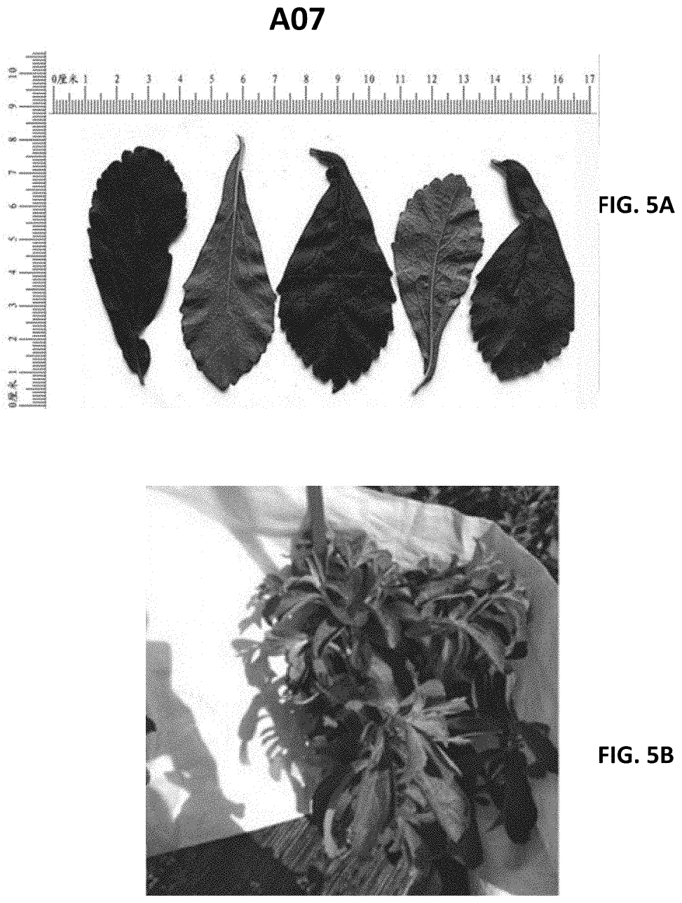

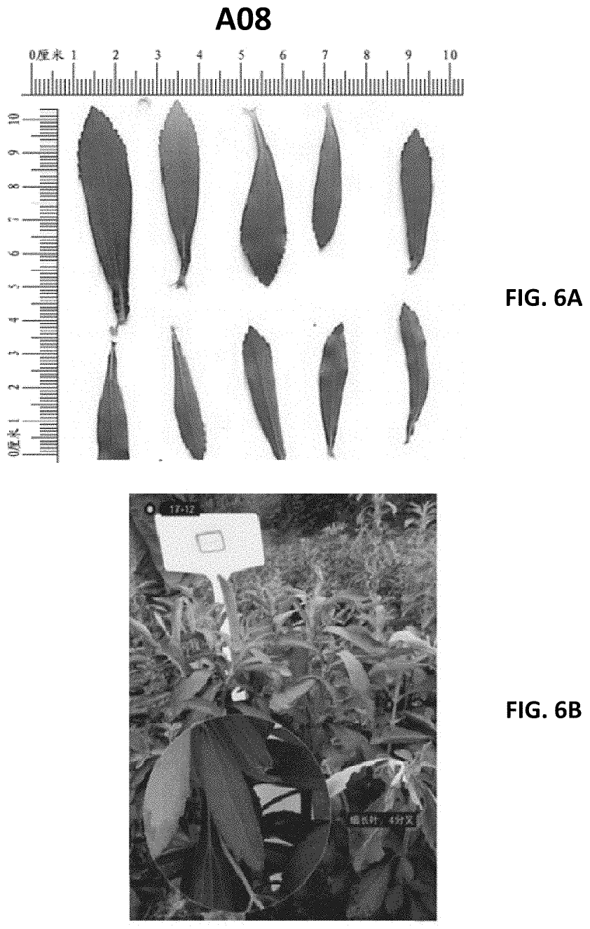

### DETAILED DESCRIPTION

The following detailed description is presented to enable any person skilled in the art to make and use the invention. The aspects of the application are described in conjunction with the exemplary embodiments, including methods, materials and examples, such description is non-limiting and the scope of the application is intended to encompass all equivalents, alternatives, and modifications, either generally known, or incorporated here. All publications, patents and patent applications cited herein are hereby incorporated by reference in their entirety.

For purposes of explanation, specific nomenclature is set forth to provide a thorough understanding of the present invention. However, it will be apparent to one skilled in the art that these specific details are not required to practice the invention.

Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### Definitions

The use of the word “a” or “an” when used in conjunction with the term “comprising” in the claims and/or the specification may mean “one,” but it is also consistent with the meaning of “one or more,” “at least one,” and “one or more than one.”

The term “cultivar”, as used herein, refers to a cultivated variety.

The term “genotype” as used herein refers to the genetic composition of a cell or organism.

The term “plant” as used herein refers to an immature or mature whole plant, including a plant that has been processed for steviol glycosides, seed or plant parts that will produce the plant, plant cells, plant protoplasts, plant cell tissue cultures from which stevia plants can be regenerated, plant calli, plant clumps, and plant cells that are intact in plants or parts of plants, such as embryos, pollen, ovules, flowers, leaves, roots, root tips, anthers, and pistils.

The term “plant part” as used herein includes leaves, stems, roots, root tips, seed, embryo, pollen, ovules, flowers, root tips, shoots, microshoots, anthers, tissue, and cells.

The term “rebaudioside A” is used herein with reference to a steviol glycoside containing only glucose as its monosaccharide moieties, which contains four glucose molecules in total with the central glucose of the triplet connected to the main steviol structure at its hydroxyl group, and the remaining glucose at its carboxyl group forming an ester bond.

The term “rebaudioside D” is used herein with reference to an ent-kaurane diterpene glycoside isolated from Stevia rebaudiana.

The term “rebaudioside M” is used herein with reference to an ent-kaurane diterpene glycoside isolated from Stevia rebaudiana.

SNP: As used herein, the term “SNP” shall refer to a single nucleotide polymorphism.

Stevioside content: As used herein, stevioside is the percent glycoside derived from the stevia plant.

Traditional breeding techniques: Encompasses herein crossing, selfing, selection, double haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one carrot line or variety to another.

Vegetative propagation: “Vegetative reproduction” or “clonal propagation” are terms used interchangeably herein and mean the method of taking part of a plant and allowing that plant part to form at least roots where plant part is, e.g., defined as or derived from (e.g., by cutting of) leaf, pollen, embryo, cotyledon, hypocotyl, cells, nodes, protoplasts, meristematic cell, root, root tip, pistil, anther, flower, shoot tip, shoot, stem, petiole, etc. When a whole plant is regenerated by vegetative propagation, it is also referred to as a vegetative propagation.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, and pistils. Means for preparing and maintaining plant tissue culture are well known in the art. By way of example, a tissue culture comprising organs has been used to produce regenerated plants.

Stevia cultivars A01, A03, A05, A06, A07 and A08 are Stevia rebaudiana varieties, which have the morphological and physiological characteristics described in Table 1 below, have shown uniformity and stability, as reflected in their reproduction over a sufficient number of generations with careful attention to uniformity of plant type. The rebaudioside contents in stevia cultivars A01, A03, A05, A06, A07 and A08 are listed in Table 2.

TABLE 1
Morphological and physiological characteristics.

| Culti- | | Character of Stevia |
|---|---|---|
| var | Appearance | Glycosides |
| A01 | Half-circle shape of leaves; | High Reb A (RA) and total |
| | visible deep serration-like | stevia glycosides; highest |
| | lines from top to middle of | content shows up around |
| | leaves | growth of 80~90 days |
| A03 | Leaves are thick and oval | RA content is in range of |
| | shaped; length of leaves is | 9.01~11.31% |
| | short; stems are strong | |
| A05 | Long leaves; visible | |
| | serration-like lines from top | |
| | to middle of leaves | |
| A06 | Leaves are spindle form, with | High RA content |
| | a sawtooth edge, thick leaves, | (9.41~12.22%) and total |
| | distance between leaves is | glycosides (14.37~17.92%); |
| | short, stem is strong | highest content shows up |
| | | around 50~70 days |
| A07 | Big leaves; visible deep | High stevioside content |
| | serration-like lines on top of | and total stevia glycosides |
| | leaves | content; highest content |
| | | shows up around 40~50 |
| | | days |
| A08 | Thin, long leaves; there are | High Reb A and total stevia |
| | few serration-like lines on | glycosides content; highest |
| | top of leaves | content shows up around |
| | | 70~80 days |

TABLE 2
Rebaudioside content of stevia glycosides

| | | | | | | | | | | | | Moisture | Age* |
|---|---|---|---|---|---|---|---|---|---|---|---|---|---|
| No. | RM | RD | RA | TD | RF | RC | DA | RU | RB | STB | TSG | (%) | (days) |
| A01 | 0.82 | 1.36 | 14.32 | 1.52 | 0.33 | 0.69 | 1.19 | 0.08 | 0.13 | 0.07 | 19.69 | 9.5 | 85 |
| A03 | 0.13 | 0.35 | 13.62 | 2.46 | 0.23 | 1.28 | 0.05 | ND | ND | ND | 17.97 | 6.18 | 90 |
| A05 | ND | 1.68 | 13.99 | 2.51 | 0.26 | 1.9 | 0.21 | 0.11 | 0.13 | ND | 20.79 | 14.3 | 56 |
| A06 | 0.20 | 0.52 | 12.15 | 1.64 | 0.22 | 1.07 | 0.02 | ND | ND | ND | 15.60 | 6.18 | 90 |
| A07 | ND | 0.84 | 3.67 | 12.49 | 0.17 | 1.41 | 1.25 | 0.00 | 0.05 | 0.06 | 19.92 | 17.5 | 40 |
| A08 | 0.66 | 2.4 | 16.45 | 1.7 | 0.43 | 0.76 | 1.25 | 0.06 | 0.08 | 0.1 | 23.22 | 8.5 | 78 |

*Age = days after planting;
TD = stevioside;
STB = steviolbioside;
TSG = total steviol glycoside

Breeding Methods

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to popular cultivars in environments representative of the commercial target area(s) for three or more years. The lines having superiority over the popular cultivars are candidates to become new commercial cultivars. Those lines still deficient in a few traits are discarded or utilized as parents to produce new populations for further selection.

These processes, which lead to the final step of marketing and distribution, usually take from seven to twelve years from the time the first cross is made. Therefore, development of new cultivars is a time-consuming process that requires precise forward planning, efficient use of resources, and a minimum of changes in direction.

A most difficult task is the identification of individuals that are genetically superior because for most traits the true genotypic value is masked by other confounding plant traits or environmental factors. One method of identifying a superior plant is to observe its performance relative to other experimental lines and widely grown standard cultivars. For many traits a single observation is inconclusive, and replicated observations over time and space are required to provide a good estimate of a line's genetic worth.

Each year, the plant breeder selects the germplasm to advance to the next generation. This germplasm is grown under unique and different geographical, climatic, and soil conditions and further selections are then made, during and at the end of the growing season. The lines which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce, with any reasonable likelihood, the same cultivar twice by using the exact same original parents and the same selection techniques. This unpredictability results in the expenditure of large amounts of research moneys to develop superior new stevia cultivars.

Pureline cultivars of stevia are commonly bred by hybridization of two or more parents followed by selection. The complexity of inheritance, the breeding objectives, and the available resources influence the breeding method. Pedigree breeding, recurrent selection breeding, and backcross breeding are breeding methods commonly used in self-pollinated crops such as stevia. These methods refer to the manner in which breeding pools or populations are made in order to combine desirable traits from two or more cultivars or various broad-based sources. The procedures commonly used for selection of desirable individuals or populations of individuals are called mass selection, plant-to-row selection, and single seed descent or modified single seed descent. One or a combination of these selection methods can be used in the development of a cultivar from a breeding population.

Pedigree breeding is primarily used to combine favorable genes into a totally new cultivar that is different in many traits than either parent used in the original cross. It is commonly used for the improvement of self-pollinating crops. Two parents which possess favorable, complementary traits are crossed to produce an F₁ (filial generation 1). An F₂ population is produced by selfing F₂ plants. Selection of desirable individual plants may begin as early as the F₂ generation wherein maximum gene segregation occurs. Individual plant selection can occur for one or more generations. Successively, seed from each selected plant can be planted in individual, identified rows or hills, known as progeny rows or progeny hills, to evaluate the line and to increase the seed quantity, or to further select individual plants. After a progeny row or progeny hill is selected as having desirable traits, it becomes what is known as a breeding line that is specifically identifiable from other breeding lines that were derived from the same original population. At an advanced generation (i.e., F₅ or higher) seed of individual lines are evaluated in replicated testing. At an advanced stage the best lines or a mixture of phenotypically similar lines from the same original cross are tested for potential release as new cultivars.

The single seed descent procedure in the strict sense refers to planting a segregating population, harvesting one seed from every plant, and combining these seeds into a bulk, which is planted as the next generation. When the population has been advanced to the desired level of inbreeding, the plants from which lines are derived will each trace to different F₂ individuals. Primary advantages of the seed descent procedures are to delay selection until a high level of homozygosity (e.g., lack of gene segregation) is achieved in individual plants, and to move through these early generations quickly, usually through using winter nurseries.

The modified single seed descent procedures involve harvesting multiple seed (i.e., a single lock or a simple boll) from each plant in a population and combining them to form a bulk. Part of the bulk is used to plant the next generation and part is put in reserve. This procedure has been used to save labor at harvest and to maintain adequate seed quantities of the population.

Selection for desirable traits can occur at any segregating generation (F₂ and above). Selection pressure is exerted on a population by growing the population in an environment where the desired trait is maximally expressed and the individuals or lines possessing the trait can be identified. For instance, selection can occur for disease resistance when the plants or lines are grown in natural or artificially-induced disease environments, and the breeder selects only those individuals having little or no disease and are thus assumed to be resistant.

In addition to phenotypic observations, the genotype of a plant can be determined using genetic markers directed to polymorphic regions that can serve to genetically differentiate one plant variety from one another. There are many types of genetic markers and laboratory-based techniques available for the analysis, comparison, and characterization of plant genotypes. The techniques are directed to the production, detection and/or identification of Restriction Fragment Length Polymorphisms (RFLPs), Simple Sequence Repeats (SSRs—which are also referred to as Microsatellites), and Single Nucleotide Polymorphisms (SNPs). Typically, these techniques involve various applications of polymer chain reaction (PCR) technology, including conventional PCR, Arbitrarily Primed Polymerase Chain Reaction (AP-PCR), and DNA Amplification Fingerprinting (DAF), which can be used to generate, for example, Randomly Amplified Polymorphic DNAs (RAPDs), Sequence Characterized Amplified Regions (SCARs), and Amplified Fragment Length Polymorphisms (AFLPs).

The cleaved amplified polymorphic sequence (CAPS) method is an extension to the Restriction Fragment Length Polymorphism (RFLP) method, using PCR to more quickly analyse the results. Like RFLP, CAPS works on the principle that genetic differences between individuals can create or abolish restriction endonuclease restriction sites, and that these differences can be detected in the resulting DNA fragment length after digestion. In the CAPS method, PCR amplification is directed across the altered restriction site, and the products digested with the restriction enzyme. When fractionated by agarose or acrylamide gel electrophoresis, the digested PCR products will give readily distinguishable patterns of bands. Alternatively, the amplified segment can be analyzed by allele-specific oligonucleotide (ASO) probes, a process that can often be done by a simple dot blot.

A microsatellite is a tract of repetitive DNA in which certain DNA motifs (ranging in length from one to six or more base pairs) are repeated, typically 5-50 times. Microsatellites occur at thousands of locations within an organism's genome. They have a higher mutation rate than other areas of DNA leading to high genetic diversity. Microsatellites are often referred to as simple sequence repeats (SSRs) by plant geneticists. ISSR (for inter-simple sequence repeat) is a general term for a genome region between microsatellite loci. The complementary sequences to two neighboring microsatellites are used as PCR primers; the variable region between them gets amplified. The limited length of amplification cycles during PCR prevents excessive replication of overly long contiguous DNA sequences, so the result will be a mix of a variety of amplified DNA strands which are generally short but vary much in length. Sequences amplified by ISSR-PCR can be used for DNA fingerprinting.

Genetic markers can also be used during the breeding process for the selection of qualitative traits. For example, markers closely linked to alleles or markers containing sequences within the actual alleles of interest can be used to select plants that contain the alleles of interest during a backcrossing breeding program. The use of genetic markers in the selection process is often called Genetic Marker Enhanced Selection. Genetic markers may also be used to identify and exclude certain sources of germplasm as parental varieties or ancestors of a plant by providing a means of tracking genetic profiles through crosses as discussed more fully hereinafter.

Mutation Breeding

Mutation breeding is another method of introducing new traits into stevia varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogues like 5-bromo-uracil), antibiotics, alkylating agents (such as sulfur mustards, nitrogen mustards, epoxides, ethylenamines, sulfates, sulfonates, sulfones, or lactones), azide, hydroxylamine, nitrous acid, or acridines. After a desired trait is observed through mutagenesis the trait may then be incorporated into existing germplasm by traditional breeding techniques.

Production of Double Haploids

The production of double haploids can also be used for the development of homozygous varieties in a breeding program. Double haploids are produced by the doubling of a set of chromosomes from a heterozygous plant to produce a completely homozygous individual.

The stevia flower is monoecious in that the male and female structures are in the same flower. The crossed or hybrid seed is produced by manual crosses between selected parents. Floral buds of the parent that is to be the female are emasculated prior to the opening of the flower by manual removal of the male anthers. At flowering, the pollen from flowers of the parent plants designated as male, are manually placed on the stigma of the previous emasculated flower. Seed developed from the cross is known as first generation F₁ hybrid seed. Planting of this seed produces F₁ hybrid plants of which half their genetic component is from the female parent and half from the male parent. Segregation of genes begins at meiosis thus producing second generation F₂ seed. Assuming multiple genetic differences between the original parents, each F₂ seed has a unique combination of genes.

Other objects, features, and advantages may become apparent from the following detailed description. It should be understood, however, that the detailed description and the specific examples, while indicating specific embodiments, are given by way of illustration only, since various changes and modifications within the spirit and scope of the embodiments of the invention may become apparent to those skilled in the art from this detailed description.

Having now described the invention, the same will be illustrated with reference to certain examples, which are included herein for illustration purposes only, and which are not intended to be limiting of the invention.

### EXAMPLES

### Example 1. Creation of New Stevia rebaudiana Cultivars, A01, A03, A05, A06, A07 and A08

The plants are selections from crosses between a series of half-sibling families purchased from the Alberta Research Council in the late 2000s. Briefly, the half-sibling families were planted in an isolated field away from other stevia plants to generate F1 seeds. The F1 seeds were then planted to generate F1 plants, which were analyzed and selected for commercially beneficial phenotype, including plant topography and chemical composition; specifically individual steviol glycoside content and total steviol glycoside content and the ratio of rebaudioside A and stevioside. The selected plants were allowed to grow to maturity (approximately 90 days) and then flower and seed. Progeny were also assessed for beneficial traits and selected. Selected commercially elite plants were cloned and maintained by tissue culture.

### Example 2. The Six Cultivars are Genetically Distinguishable from Other S. rebaudiana Lines

To further establish the uniqueness of the varieties created in Example 1, molecular testing was conducted on a wide range of varieties from publically available entities and from commercial companies.

Briefly, RNA was extracted from germplasms corresponding to the following population groups of stevia lines: (1) SGF lines: A01, A03, A05, A06, A07 and A08; (2) doubled haploid (DH) lines from SGF: T54, T55, T58 and T60; (3) control lines (CL): CL1, CL2 and CL3; and (4) publicly available (Public) material that was previously sequenced was downloaded from NCBI: SRR5059304, SRR5059305, SRR5059306, SRR5059307 and SRR5059308.

RNA was sequenced at Novogene LLC (San Diego, Calif.). Raw sequence reads were aligned to the publicly available transcriptome GANE (https://www.ncbi.nlm.nih.gov/bioproject/209090) using bowtie (Langmead, B., & Salzberg, S. L. (2012) Nature methods, 9(4), 357). Single nucleotide polymorphisms were called using NGSEP (Tello, D., et al. (2019) Bioinformatics, 35(22), 4716-4723). This resulted in the identification of 31,248 single nucleotide polymorphism (SNP) markers used for the divergence/differentiation analysis that follows.

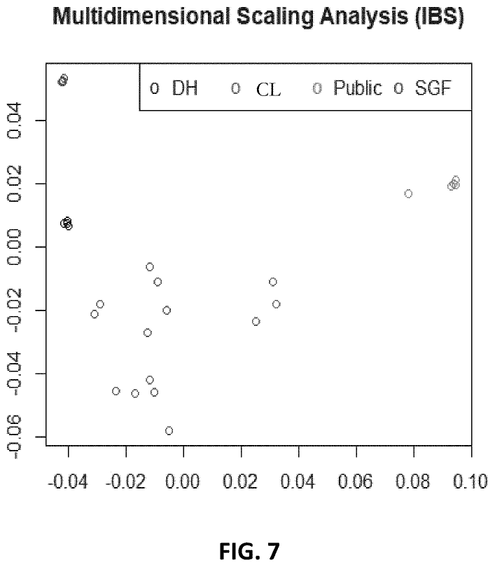

Multi-dimensional scaling was employed to determine the degree of genetic divergence between individual groups of stevia varieties (i.e., germplasms). This analysis was conducted using the “identity-by-state function of the SNPrelate (Zheng, X., et al. (2012) Bioinformatics, 28(24), 3326-3328) for each individual group. As shown in FIG. 7, the multi-dimensional scaling clearly differentiated the group of SGF varieties (i.e., A01, A03, A05, A06, A07 and A08) from the foregoing group of previously patented doubled haploid (DH) lines in Group (2) and control lines (CL) in Group (3) (above). This is reflected in the separate clusters in the genetic space corresponding to the different population groups of stevia varieties (SGF=blue, DH=black, Public=green, and CL=red).

Based on the RNA sequences obtained or collected, diversity statistics were calculated and FsT population differentiation was determined using Hierfstat (Goudet, J. (2005) Molecular Ecology Notes, 5(1), 184-186). The results of this analysis are shown in Table 3. Analysis of molecular variance and calculation of basic diversity statistics was conducted using Popp (Kamvar, Z. N., Tabima, J. F., & Grunwald, N. J. (2014) PeerJ, 2, e281). Pairwise genetic distances were calculated using Adegenet (Jombart, T., & Ahmed, I. (2011) Bioinformatics, 27(21), 3070-3071).

Significant differentiation at the alpha=0.01 level between the different groups of varieties was observed (Table 3). In this case, the SGF group showed the highest level of diversity (Table 4). Further, an analysis of molecular variance that showed significant variation between the different population groups (Table 5). All stevia varieties were clearly differentiated based on pairwise genetic distances (Table 6).

TABLE 3
Weir & Cockerham Fst values between different
stevia population groups.

| | DH | CL | Public |
|---|---|---|---|
| CL | 0.13 | | |
| Public | 0.16 | 0.09 | |
| SGF | 0.08 | 0.03 | 0.09 |

TABLE 4
Diversity statistics pertaining to different stevia
population groups.

| Popula- | Hetero- | Shannon | Stoddard and | Simpson |
|---|---|---|---|---|
| tion | zygosity | Index | Taylor Index | lambda |
| DH | 0.27 | 1.39 | 4.00 | 0.75 |
| CL | 0.29 | 1.10 | 3.00 | 0.67 |
| Public | 0.28 | 1.61 | 5.00 | 0.80 |
| SGF | 0.31 | 1.79 | 6.00 | 0.83 |

TABLE 5
AMOVA tests of significance for the different
tests of variation.

| Test | Obs | Std. Obs. | Alter | P-value |
|---|---|---|---|---|
| Variation within an | 8060.3994 | 4.1103 | less | 1 |
| individual variety | | | | |
| Variation between | −3572.078 | −3.423628 | greater | 1 |
| varieties in a given | | | | |
| population group | | | | |
| Variation between | 477.4242 | 9.095288 | greater | 0.001 |
| different population | | | | |
| groups | | | | |

TABLE 6
Pairwise genetic distances.

| | CL1 | CL2 | A01 | A03 | A05 | A06 | A07 | A08 | CL3 |
|---|---|---|---|---|---|---|---|---|---|
| CL2 | 110.99 | | | | | | | | |
| A01 | 121.72 | 122.73 | | | | | | | |
| A03 | 121.33 | 114.77 | 105.25 | | | | | | |
| A05 | 124.8 | 121.22 | 112.52 | 106.61 | | | | | |
| A06 | 120.78 | 119.39 | 106.18 | 102.47 | 104.45 | | | | |
| A07 | 125.58 | 126.23 | 110.88 | 111.9 | 116.45 | 116.09 | | | |
| A08 | 121.57 | 119.84 | 115.1 | 118.95 | 113.55 | 114.03 | 112.86 | | |
| CL3 | 114.66 | 114.91 | 122.62 | 119.72 | 124.98 | 121.31 | 121.33 | 120.35 | |
| SRR5059304 | 118.87 | 120.12 | 127.03 | 125.27 | 132.06 | 12183 | 131.28 | 123.7 | 117.04 |
| SRR5059305 | 119.25 | 119.89 | 128.45 | 125.54 | 132.24 | 124.52 | 131.81 | 124.01 | 117.14 |
| SRR5059306 | 119.74 | 119.53 | 128.38 | 125.67 | 132.3 | 124.61 | 131.42 | 124.42 | 117.23 |
| SRR5059307 | 118.94 | 120.5 | 127.83 | 126.27 | 132.25 | 123.25 | 131.71 | 124.14 | 117.71 |
| SRR5059308 | 120.44 | 118.69 | 126.93 | 126.75 | 129.24 | 123.24 | 131.38 | 123.26 | 116.99 |
| T54 | 123.89 | 119.55 | 112.27 | 114.97 | 112.74 | 108.38 | 114.18 | 110.51 | 121.35 |
| T55 | 123.76 | 119.32 | 112.1 | 114.78 | 112.66 | 108.4 | 114.15 | 110.76 | 121.59 |
| T58 | 124.74 | 119.98 | 112.77 | 115.69 | 113.28 | 109.24 | 115.17 | 111.45 | 122.57 |
| T60 | 124.07 | 119.98 | 111.47 | 115.46 | 113.34 | 108.93 | 114.42 | 110.74 | 121.84 |
| | Pairwise genetic distances. | | | | | | | | |
| | | SRR5059304 | SRR5059305 | SRR5059306 | SRR5059307 | SRR5059308 | T54 | T55 | T58 |
| | CL2 | | | | | | | | |
| | A01 | | | | | | | | |
| | A03 | | | | | | | | |
| | A05 | | | | | | | | |
| | A06 | | | | | | | | |
| | A07 | | | | | | | | |
| | A08 | | | | | | | | |
| | CL3 | | | | | | | | |
| | SRR5059304 | | | | | | | | |
| | SRR5059305 | 19.07 | | | | | | | |
| | SRR5059306 | 18.79 | 17.09 | | | | | | |
| | SRR5059307 | 16.63 | 22.46 | 20.91 | | | | | |
| | SRR5059308 | 90.55 | 90.34 | 90.65 | 90.93 | | | | |
| | T54 | 126.96 | 127.81 | 127.71 | 127.53 | 126.23 | | | |
| | T55 | 126.93 | 127.97 | 127.94 | 127.48 | 126.39 | 16.81 | | |
| | T58 | 127.79 | 128.72 | 128.36 | 128.17 | 126.92 | 17.69 | 18.97 | |
| | T60 | 127.38 | 127.94 | 127.96 | 127.86 | 126.39 | 14.5 | 17.66 | 17.68 |

### Example 3. Development and Validation of Markers that Differentiate the Six Cultivars Described Herein from Other S. rebaudiana Lines

Currently there are ˜1000 commercially available cultivars, thus it is necessary be able to differentiate among this number of cultivars. Typically, each individual cultivar has two alleles at a locus because stevia is a diploid organism. When a cultivar contains the same allele (e.g., A/A or BB) at a locus, it is homozygous at that locus; when it contains different alleles (e.g., AB) at a locus, the cultivar is heterozygous at that locus. If there are more than two alleles at a locus, the additional alleles are expressed as allele “C,” “D,” and so on. A multi-locus genotype is the combination of individual locus genotypes across multiple loci, which provides a means for distinguishing between different varieties.

The most commonly used marker type are single nucleotide polymorphisms (SNPs). However, these markers are hard to visualize on agarose gels. Depending on whether the sequences surrounding a given SNP specify a specific restriction enzyme (RE) cut site, some SNP markers can be utilized in the context of cleaved amplified polymorphic sequence (CAPS) and one or more restriction enzymes (REs) to create an easily ascertainable co-dominant marker for genotyping. Another type of co-dominant marker is the presence of insertions or deletions (INDEL) within a polymorphic locus. Both CAPS and INDEL markers are PCR based markers that are easy to use, robust in nature, providing an effective and relatively inexpensive means for easily determining the presence or absence of alleles at a given locus.

Based on the sequence information obtained in Example 2, a set of CAPS/PCR/ISSR markers was developed in this example and further validated for purposes of differentiating SGF proprietary varieties A01, A03, A05, A06, A07 and A08 from the other stevia varieties. Table 7 shows the marker loci, primer sets, amplicon sizes, restriction enzymes, and annealing temperatures for use in differentiating stevia cultivars according to the present application as further exemplified in Example 4.

TABLE 7
Genetic markers for differentiating stevia cultivars.

| | | | Type | | | Restr. | |
|---|---|---|---|---|---|---|---|
| | | | of | | | Enz. | Annealing |
| No. | Locus | HARC ID | Marker | Primer-F | Primer-R | (RE) | Temp. (° C.) |
| 1 | GANE01021955 | JD007_08 | CAPS | CGGAACCTCGGA | AAGAGGAAGAAT | AvaI | 60 |
| | | | | ATCATCTT | GAAGAGAAGAGG | | |
| | | | | (SEQ ID | (SEQ ID | | |
| | | | | NO: 1) | NO: 2) | | |
| 2 | GANE01016707 | JD005_06 | CAPS | CCTTCGATAGTTC | CAATCTCACTAG | DraI | 60 |
| | | | | TGACGCTTAC | GAAGGGTTCG | | |
| | | | | (SEQ ID | (SEQ ID | | |
| | | | | NO: 3) | NO: 4) | | |
| 3 | GANE01016455 | JD009_10 | PCR | GAAAGCTACCGG | TACATCGGAATC | N/A | 62 |
| | | | | AGCAGATAAA | AGTCCCGA | | |
| | | | | (SEQ ID | (SEQ ID | | |
| | | | | NO: 5) | NO: 6) | | |
| 4 | GANE01004406 | JD011_12 | CAPS | ACAAGTCATGATC | CGTTCTTCCTCCT | HphI | 65-->55 |
| | | | | AGACCATCTT | AGCTTCTTC | | (10 cycles) |
| | | | | (SEQ ID | (SEQ ID | | 55 (25 cycles) |
| | | | | NO: 7) | NO: 8) | | |
| 5 | GANE01025251 | JD013_14 | CAPS | CCTTCGATAGTTC | CAATCTCACTAG | AluI | 60 |
| | | | | TGACGCTTAC | GAAGGGTTCG | | |
| | | | | (SEQ ID | (SEQ ID | | |
| | | | | NO: 9) | NO: 10) | | |
| 6 | GANE01000877 | JD025_26 | CAPS | ACCCAAGAACCC | AGAGACAAACCT | HphI | 65-->55 |
| | | | | GAATCAGG | ACCCAGTGA | | (10 cycles) |
| | | | | (SEQ ID | (SEQ ID | | 55 (25 cycles) |
| | | | | NO: 11) | NO: 12) | | |
| 7 | GANE01003004 | JD027_28 | CAPS | ACGGTGAACTGC | GATTTGTGGCCG | HhaI | 61 |
| | | | | GCTGATAA | AGGATTGC | | |
| | | | | (SEQ ID | (SEQ ID | | |
| | | | | NO: 13) | NO: 14) | | |
| 8 | GANE01011595 | JD033_34 | CAPS | ATTCCGCACTACC | TGAAAGACCGAA | EcoP15I | 65 (5 cycles), |
| | | | | ATACGCC | TGCAATACGC | | 65-->60 |
| | | | | (SEQ ID | (SEQ ID | | (5 cycles) |
| | | | | NO: 15) | NO: 16) | | 60 (25 cycles) |
| 9 | GANE01015568 | JD037_38 | CAPS | TCACGATCGTTTG | CTGACCCCATCTT | BamHI | 65-->60 |
| | | | | GAACCGT | CCCCAAC | BstYI | (5 cycles |
| | | | | (SEQ ID | (SEQ ID | | 60 (30 cycles) |
| | | | | NO: 17) | NO: 18) | | |
| 10 | GANE01016432 | JD039_40 | PCR | ATGTCGCTGTTTC | CTTTGTGGGGCTT | N/A | 64 |
| | | | | TTCGGGT | CAGCAAC | | (35 cycles)** |
| | | | | (SEQ ID | (SEQ ID | | |
| | | | | NO: 19) | NO: 20) | | |
| 11 | WOUH023266.1 | JD059_60 | CAPS | TGGAACTACAATC | CGAGTCCTTCGT | AvaI | 65-->60 |
| | | | | CCTCCAATC | AGACGTTAAT | | (5 cycles) |
| | | | | (SEQ ID | (SEQ ID | | 60 (30 cycles) |
| | | | | NO: 21) | NO: 22) | | |
| 12 | WOUH023266.1.2 | JD059_60 | CAPS | TGGAACTACAATC | CGAGTCCTTCGT | HphI | 65-->60 |
| | | | | CCTCCAATC | AGACGTTAAT | | (5 cycles) |
| | | | | (SEQ ID | (SEQ ID | | 60 (30 cycles) |
| | | | | NO: 23) | NO: 24) | | |
| 13 | WOUH01005557.1 | JD065B_66B | CAPS | CGGAAGTCCTCTA | GATTGAAAGAGC | HhaI | 56.3 |
| | | | | GTGTTTAAG | CGAGGTATC | | |
| | | | | (SEQ ID | (SEQ ID | | |
| | | | | NO: 25) | NO: 26) | | |
| 14 | UDP74G1 | UDP74G1-F | CAPS | AATCGGGCCAAC | AGTCGCAAAATT | Alu | 65-->60 |
| | | UDP74G1-R | | ACTTCCAT | TTAGGACAAAGA | | (5 cycles) |
| | | | | (SEQ ID | (SEQ ID | | 60 (30 cycles) |
| | | | | NO: 27) | NO: 28) | | |
| 15 | DVE44 | N/A | ISSR | AGAGAGAGAGAG | N/A | N/A | 55* |
| | | | | AGAGC | | | |
| | | | | (SEQ ID | | | |
| | | | | NO: 29) | | | |
| 16 | UDP85C2 | JD069-F | PCR | CCACATTGTCTAG | CGGTGATTTCCTT | N/A | 62 |
| | | JD072-R | | ACGGTTC | GACCATTT | | |
| | | | | (SEQ ID | (SEQ ID | | |
| | | | | NO: 30) | NO: 31) | | |

*For ISSR, 10 second extension for 2 kb cut-off (using fast polymerase), all other PCR conditions (unless otherwise noted) use an extension time of 15 seconds. All PCRs use a final extension time of 3 minutes.
**Initial denature 2.5 min; subsequent cycles: denature 20 s; anneal 15 s; extend 25 s.

### Example 4. Validation of Genetic Markers for Genotyping S. rebaudiana Cultivars and Differentiating them from Each Other and Other S. rebaudiana Lines

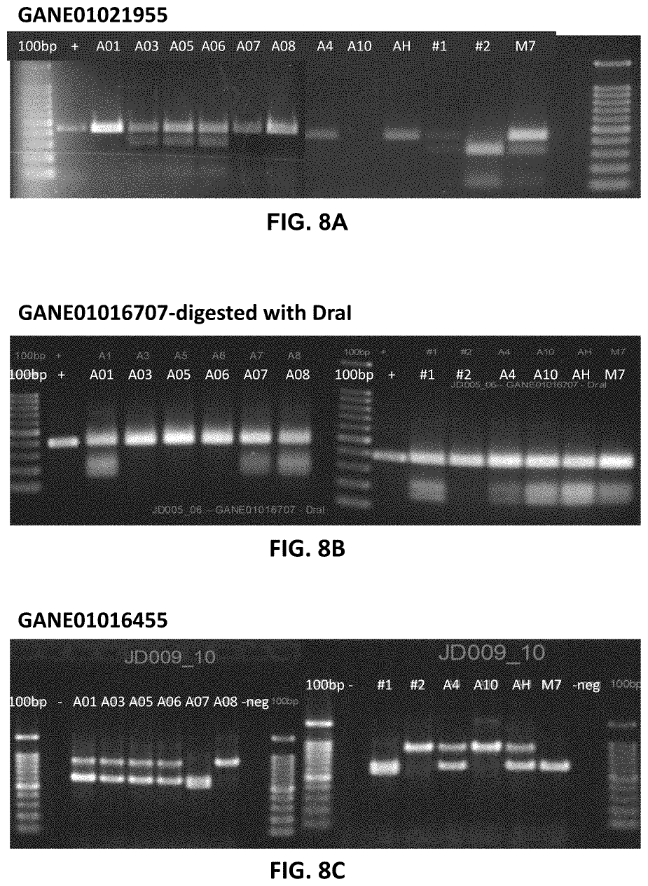

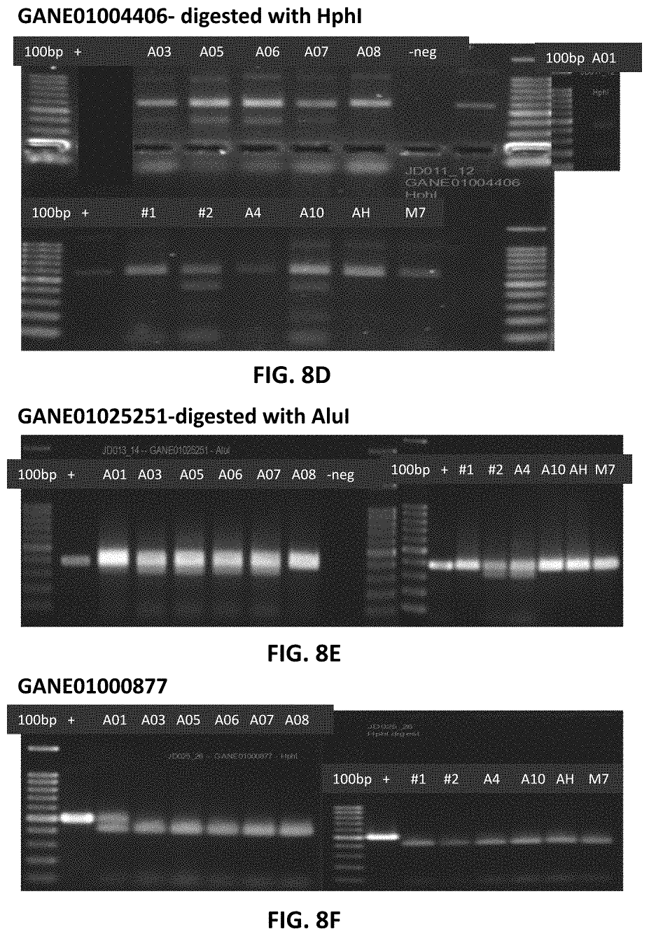

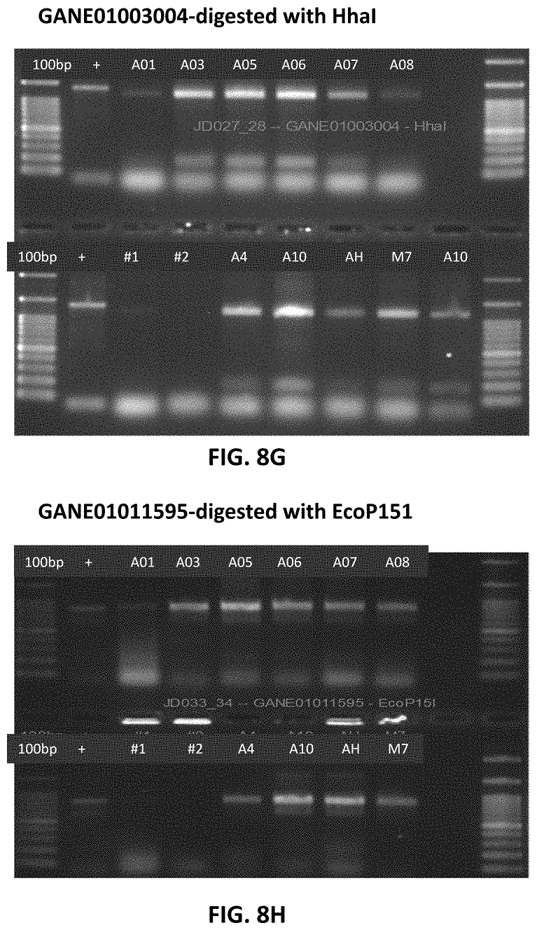

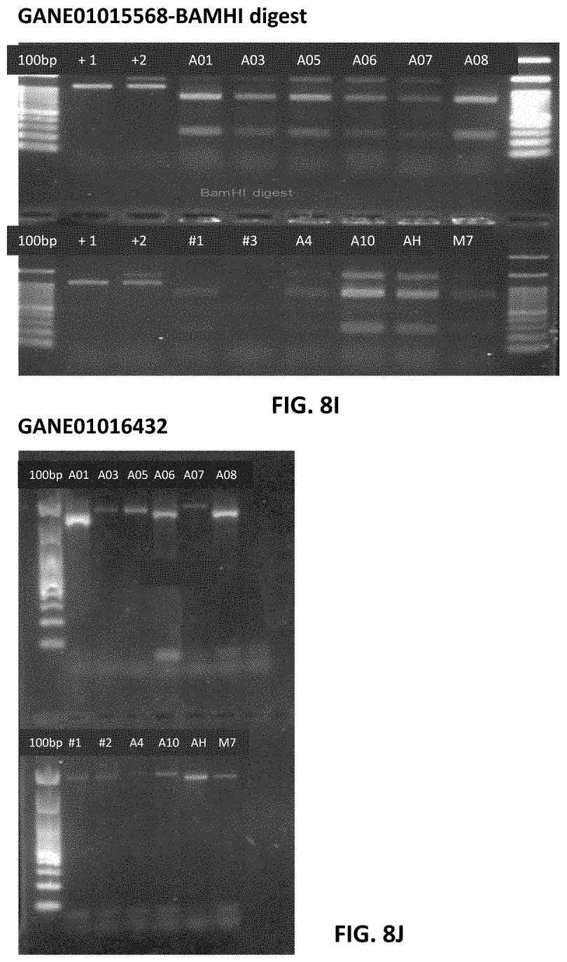

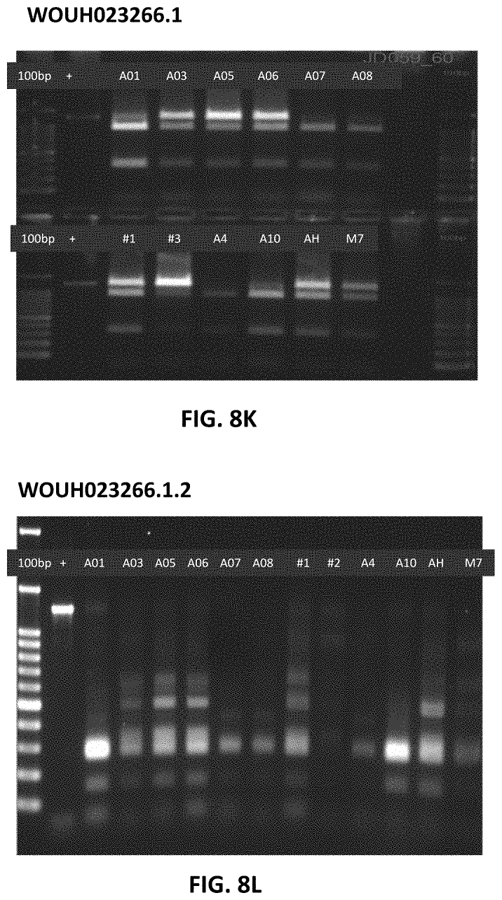

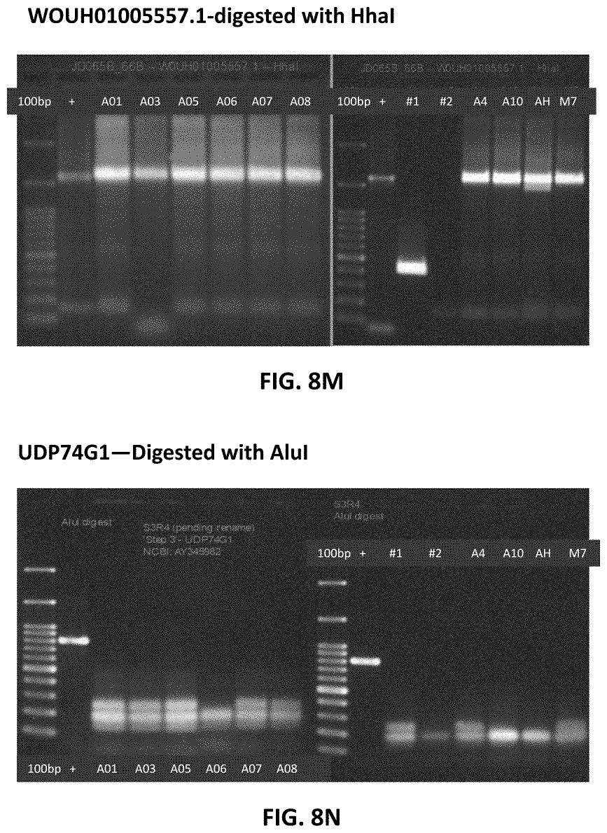

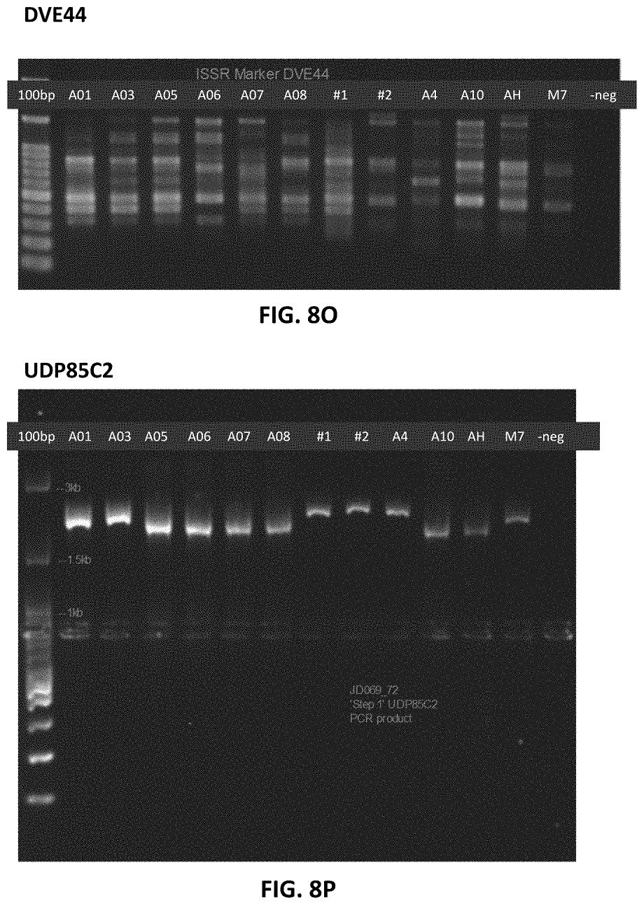

The markers described in Table 7 of Example 3 were tested for their ability to genotype and differentiate SGF cultivars A01, A03, A05, A06, A07 and A08 from each other and other stevia cultivars. As further described below with reference to Table 8 and FIGS. 8A-8P, the utility of these markers was validated for use in genotyping the SGF stevia cultivars and differentiating these cultivars from one another, as well stevia cultivars from different sources (commercial, wild, public). Validation of these markers was evidenced by their use in producing consistent banding patterns on agarose gels characteristic for a given cultivar. Using these markers, stevia cultivars can be genotyped by any lab with access to a PCR machine and gel electrophoresis.

Validation of the markers for genotyping stevia cultivars was carried out as follows. Briefly, DNA from a series of stevia cultivars (including SGF cultivars A01, A03, A05, A06, A07, A08 and control cultivars (e.g., #1, #2, M7, A4, A10 and AH) was extracted and amplified using the primers described in Table 7. The DNAs were extracted from dried, fresh or frozen stevia samples of stevia leaves and stems using the following modified protocol developed from the methods and reagents utilized in the DNeasy Blood and Tissue Kit (QIAGEN Inc., Valencia, Calif.), and the TrimGen DNA Extraction Kit (TRIMGEN Corp., Sparks, Md.).

DNA Extraction Protocol.

1) Pre-warm heating block or water bath to 60 C, 55 C and 95 C, in that order. Set the heating block to 95 C between step 13 and 14.

2) Collect 1-3 stevia leaves (˜100 mg fresh tissue)

a. If liquid nitrogen is available, dip and store the samples in liquid nitrogen until ready for use

b. Pulverize the sample

3) Add 400 μl Buffer AP1 and 4 μl RNase A (in that order) to the same tube with pulverized tissue

4) Vortex and incubate for 10 min at 65° C.

5) Invert the tube 2-3 times during incubation

6) Add 130 μl Buffer P3

a. Mix and incubate of 5 min on ice

7) Centrifuge the lysate for 5 min at 20,000×g (14,000 rpm)

8) Pipet the lysate into a Qiagen QIAshredder spin column placed in a 2 ml collection tube.

9) Centrifuge for 2 min at 20,000×g

10) Transfer the flow-through into a new tube without disturbing the pellet if present

11) Add 1 ml of TrimGen Wash Buffer to the tube

12) Vortex 10 s at maximum speed (20000 g)

13) Centrifuge the tube at 10,000×g for 10 minutes.

14) Discard the supernatant using a pipette or by aspiration. Be careful not to disturb the pellets, which are clear and difficult to see and located on the bottom and the sides of the tube.

15) Re-suspend TrimGen WaxFree Resin by shaking the bottle several times. Transfer 120 ul of the WaxFree Resin to each tube

16) Add 7 ul of TrimGen Enzyme Mix to each tube

17) Mix the contents of the tube by flicking the tube with a gloved finger

18) Incubate the tube at 55 C for 60 minutes, (If the sample is low quality or old—a longer incubation time of 3 hours to overnight can be used)

19) For best results, flick the tubes by finger several times during this incubation

20) Heat the tube at 95 C for 10 minutes

21) Place TrimGen WR-Filter into a new tube (make sure that the white filter is at the bottom of the column and properly set as the WR-filter removes undigested tissue and WaxFree resin. After the final spin, the final extract may look cloudy, however, it will not affect the PCR reaction.)

22) After incubation, transfer entire extraction mix onto the WR filter

23) Centrifuge at 1,000×g for 2 minutes

24) Discard the WR-Filter. The solution in the tube is the final extract containing DNA, which is ready for PCR amplification. Typical yield is 150-500 ng/μl.

PCR Amplification

To detect the above-described polymorphic markers in the stevia cultivars, the extracted DNAs were subjected to PCR amplification in accordance with the following PCR conditions, which were developed using MyTaq™ HS Red Mix (BioLine USA Inc./Meridian Bioscience), a pre-mixed master mix (2×) containing all necessary reagents and requiring only water, template and primer as additional input. The speed of the Taq polymerse in this master mix was experimentally determined for fragments under 2 kb to be ˜133 nt/sec (i.e. 15 s extension time for a 1.7 kb amplicon). It should be noted that not all master mixes exhibit this speed, especially for Taq, and that some adjustments may be needed.

In general, for each of the amplicons described in Table 7, a polymerase chain reaction (PCR) was performed in a total volume of 50 μl containing 2-15 ng of template DNA, 2 μL from a 10 μM primer stock solution, 25 μL of MyTaq™ HS Red Mix, 2×, and water (up to Except for certain modifications to the following general PCR reaction protocol, including those described in Table 7, the PCR reactions used the following cycling conditions: initial denaturation at 95° C. for 3 min; followed by 35 cycles (95° C. for 15 sec, variable annealing ° C. for 15 sec, extension at 72° C. for 15 sec); and an optional final extension of 4 min at 72° C.

Visualization of Amplification Products

To characterize and visualize amplification products directly, or those subsequently digested with the restriction enzymes described in Table 7, the following visualization protocol was employed: (1) Make a 1-2% agarose gel using high heat agarose (appropriate for standard gel electrophoresis); (2) Electrophorese the reaction products at a voltage equal to 5-10 V/cm (cm measured as the distance between electrodes); (3) Stain the agarose gel with GelRed at 15 ul/50 ml agar for 30 min; (4) Expose the gel to UV light to visualize the bands; and (5) Photograph the gel under UV light to facilitate determination of the corresponding genotype.

Table 8 depicts genotypes for the stevia varieties evaluated, including the SGF varieties, A01, A03, A05, A06, A07, and A08, based on the absence (“A”) or presence (“B”) of the indicated restriction site in the CAPS markers listed in the table. Table 8 further identifies the marker types associated with each polymorphic locus, as well as the restriction enzymes (REs) used, fragment sizes corresponding to the A and B alleles, and the application figure number exemplifying use of the specific marker to genotype the corresponding polymorphic locus.

TABLE 8
Marker genotyping of S. rebaudiana lines.

| | | | Ampli- | | | | | | | | | | |
|---|---|---|---|---|---|---|---|---|---|---|---|---|---|
| | Marker | | con | | | | | | | | | | Fig. |
| Locus | Type | RE | Size | A01 | A03 | A05 | A06 | A07 | A08 | #1 | #2 | M7 | No. |
| GANE01021955 | CAPS | AvaI | 450 | A | A | A | A | A | A | A | B | A | 8A |
| | | | | A | B | B | B | A | A | B | B | B | |
| GANE01016707 | CAPS | .DraI | 400 | A | A | A | A | A | A | A | A | A | 8B |
| | | | | B | A | A | A | B | B | B | A | B | |
| GANE01016455* | PCR | n/a | 800- | A | A | A | A | B | A | B | A | B | 8C |
| | | | 100 | B | B | B | B | B | A | B | A | B | |
| GANE01004406 | CAPS | HphI | 600 | A | A | A | A | A | A | A | A | A | 8D |
| | | | | A | B | B | B | B | A | A | B | A | |
| GANE01025251 | CAPS | AluI | 400 | A | A | A | A | A | A | A | A | A | 8E |
| | | | | A | A | A | A | A | A | A | B | A | |
| GANE01000877 | CAPS | HphI | 500 | A | B | B | B | B | B | B | B | B | 8F |
| | | | | B | B | B | B | B | B | B | B | B | |
| GANE01003004 | CAPS | HhaI | 1300 | A | A | A | A | A | A | A | n/a | A | 8G |
| | | | | A | B | B | B | B | A | A | | B | |
| GANE01011595 | CAPS | EcoP15I | 750 | B | A | A | A | A | A | n/a | n/a | A | 8H |
| | | | | B | A | A | A | A | A | | | A | |
| GANE01015568 | CAPS | BatnHI | 1100- | B | A | A | A | B | B | B | A | B | 8I |
| | | .BstYI | 1400 | B | B | B | B | B | B | B | B | B | |
| GANE01016432* | PCR | n/a | 1800- | B | A | A | A | n/a | A | A | A | B | 8J |
| | | | 2000 | B | A | A | A | | A | A | A | B | |
| WOUH023266.1 | CAPS | AvaI | 1300 | B | A | A | A | B | B | A | A | A | 8K |
| | | | | B | B | B | B | B | B | B | A | B | |
| WOUH023266.1.2 | CAPS | HphI | 1300 | B | A | A | A | B | B | A | n/a | B | 8L |
| | | | | B | B | B | B | B | B | B | | B | |
| WOUH01005557.1 | CAPS | HhaI | 1600 | A | A | A | A | A | A | B | n/a | A | 8M |
| | | | | A | A | A | A | A | A | B | | A | |
| UDP74G1 | CAPS | AluI | 800 | A | A | A | B | A | A | A | B | A | 8N |
| | | | | B | B | B | B | B | B | B | B | B | |
| DVE44** | ISSR | n/a | n/a | 3 | 4 | 5 | 5 | 4 | 4 | 3 | 3 | 2 | 8O |
| UDP85C2* | PCR | n/a | 1800 | A | A | B | B | B | B | A | A | A | 8P |
| | | | | A | A | B | B | B | B | A | A | A | |

*Alleles in these markers are identified by size of the amplicon: A = large amplicon, B = small amplicon.
**This is an ISSR marker, the alleles are distinguishable by the band patterns of the PCR product.

In Table 8, each allele for a given locus in a particular stevia variety is defined as “A” or “B” in accordance with the following conditions. Where the marker in Table 7 is a CAPS marker, a designation of “A” corresponds to an allele in which production of the amplicon by PCR and subsequent digestion results in the production of a fragment that is the same size of the amplicon, consistent with the absence of the particular restriction enzyme site in the amplicon as described in Table 7 (i.e., not digested). By contrast, when using a CAPS marker, a designation of “B” corresponds to an allele in which PCR amplification followed by digestion with a restriction enzyme corresponding to a CAPS marker described in Table 7 produces a fragment(s) that is reduced in size, consistent with the presence of that particular restriction enzyme site in the amplicon (i.e. digested). Therefore, a designation of A/A for a particular marker means that both alleles at the marker position do not contain the corresponding restriction enzyme site. A designation of BB for a particular marker means that both alleles at the marker position contain the corresponding restriction enzyme site. A designation of AB for a particular marker means that one allele at the marker position contains the corresponding restriction enzyme site, while the other allele at the marker position does not contain the corresponding restriction enzyme site.

Where the marker in Table 7 is designated by the term “PCR”, the “A” and “B” alleles for the corresponding locus can be determined on the basis of characteristic amplicon sizes (in bp) obtained from the amplicon product produced from the corresponding primers described in Table 7. In this case, the different fragment sizes correspond to differences in the number of repetitive sequence elements within the region producing the amplicon.

In Table 7, the marker corresponding to the DVE44 locus is used to detect a type of simple sequence repeat (SSR) known as an “inter-simple sequence repeat” (ISSR). An ISSR is a general term for a genome region between microsatellite loci. In this case, complementary sequences to two neighboring microsatellites are used as PCR primers; the variable region constitutes the bulk of what is amplified in the PCR reaction. The limited length of amplification cycles during PCR prevents excessive replication of overly long contiguous DNA sequences, so the result will be a mix of a variety of amplified DNA strands which are generally short but vary enough in length to allow for differentiation of stevia varieties that are polymorphic in this region.

DNA fragments amplified or extended via PCR amplification (or PCR extension) were subjected to digestion (or not) with the restriction enzymes in Tables 7 and 8, as indicated. The resulting DNA fragments were then separated on agarose gels by electrophoresis as shown in FIGS. 8A-8P to determine the genotypes corresponding to a given stevia variety. The results in FIGS. 8A-8P serve to validate the use of the genetic markers characterized herein and provide means for genetically distinguishing the SGF varieties produced herein from one another and from other publicly available stevia varieties described herein.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the claimed components and steps in any sequence which is effective to meet the objectives there intended, unless the context specifically indicates the contrary.

What is claimed is:

1. A stevia plant, comprising the allele combination at genetic marker sites listed below:
A/A or A/B at genetic marker site GANE01021955;
A/A or A/B at genetic marker site GANE01016707;
A/A or A/B at genetic marker site GANE01004406;
A/B or B/B at genetic marker site WOUH023266.1
A/A at genetic marker site GANE01025251;
A/A or B/B at genetic marker site UDP85C2; and
A/A at genetic marker site WOUH01005557.1, with the proviso that when GANE01021955 is A/B, GANE01004406 is A/B.

2. The stevia plant of claim 1, further comprising one or more of the alleles selected from the group consisting of:
A/B or B/B at genetic marker site GANE01000877;
A/A or A/B at genetic marker site GANE01003004;
A/A or B/B at genetic marker site GANE01011595;
A/B or B/B at genetic marker site GANE01015568;
A/B or B/B at genetic marker site WOUH023266.1.2;
A/B or B/B at genetic marker site UDP74G1; and
A/A or B/B at genetic marker site GANE01016432.

3. The stevia plant of claim 2, further comprising the allele of A/B or B/B at genetic marker site GANE01000877.

4. The stevia plant of claim 3, further comprising the allele of A/A or A/B at genetic marker site GANE01003004.

5. The stevia plant of claim 4, further comprising allele A/A or B/B at genetic marker site GANE01011595.

6. The stevia plant of claim 5, further comprising allele A/B or B/B at genetic marker site GANE01015568.

7. The stevia plant of claim 6, further comprising allele A/B or B/B at genetic marker site WOUH023266.1.2.

8. The stevia plant of claim 7, further comprising allele A/B or B/B at genetic marker site UDP74G1.

9. The stevia plant of claim 8, further comprising allele A/A or B/B at genetic marker site GANE01016432.

10. The stevia plant of claim 1, wherein the plant comprises:
A/A at genetic marker site GANE01021955;
A/B at genetic marker site GANE01016707;
A/B at genetic marker site GANE01016455;
A/A at genetic marker site GANE01004406;
A/A at genetic marker site GANE01025251;
A/B at genetic marker site GANE01000877;
A/A at genetic marker site GANE01003004;
B/B at genetic marker site GANE01011595;
B/B at genetic marker site GANE01015568;
B/B at genetic marker site GANE01016432;
B/B at genetic marker site WOUH023266.1;
B/B at genetic marker site WOUH023266.1.2;
A/A at genetic marker site WOUH01005557.1;
A/B at genetic marker site UDP74G1; and
A/A at genetic marker site UDP85C2.

11. The stevia plant of claim 1, wherein the plant comprises:
A/B at genetic marker site GANE01021955;
A/A at genetic marker site GANE01016707;
A/B at genetic marker site GANE01016455;
A/B at genetic marker site GANE01004406;
A/A at genetic marker site GANE01025251;
B/B at genetic marker site GANE01000877;
A/B at genetic marker site GANE01003004;
A/A at genetic marker site GANE01011595;
A/B at genetic marker site GANE01015568;
A/A at genetic marker site GANE01016432;
A/B at genetic marker site WOUH023266.1;
A/B at genetic marker site WOUH023266.1.2;
A/A at genetic marker site WOUH01005557.1;
A/B at genetic marker site UDP74G1; and
A/A at genetic marker site UDP85C2.

12. The stevia plant of claim 1, wherein the plant comprises:
A/B at genetic marker site GANE01021955;
A/A at genetic marker site GANE01016707;
A/B at genetic marker site GANE01016455;
A/B at genetic marker site GANE01004406;
A/A at genetic marker site GANE01025251;
B/B at genetic marker site GANE01000877;
A/B at genetic marker site GANE01003004;
A/A at genetic marker site GANE01011595;
A/B at genetic marker site GANE01015568;
A/A at genetic marker site GANE01016432;
A/B at genetic marker site WOUH023266.1;
A/B at genetic marker site WOUH023266.1.2;
A/A at genetic marker site WOUH01005557.1;
A/B at genetic marker site UDP74G1; and
B/B at genetic marker site UDP85C2.

13. The stevia plant of claim 1, wherein the plant comprises:
A/B at genetic marker site GANE01021955;
A/A at genetic marker site GANE01016707;
A/B at genetic marker site GANE01016455;
A/B at genetic marker site GANE01004406;
A/A at genetic marker site GANE01025251;
B/B at genetic marker site GANE01000877;
A/B at genetic marker site GANE01003004;
A/A at genetic marker site GANE01011595;
A/B at genetic marker site GANE01015568;
A/A at genetic marker site GANE01016432;
A/B at genetic marker site WOUH023266.1;
A/B at genetic marker site WOUH023266.1.2;
A/A at genetic marker site WOUH01005557.1;
B/B at genetic marker site UDP74G1; and
B/B at genetic marker site UDP85C2.

14. The stevia plant of claim 1, wherein the plant comprises:
A/A at genetic marker site GANE01021955;
A/B at genetic marker site GANE01016707;
B/B at genetic marker site GANE01016455;
A/B at genetic marker site GANE01004406;
A/A at genetic marker site GANE01025251;
B/B at genetic marker site GANE01000877;
A/B at genetic marker site GANE01003004;
A/A at genetic marker site GANE01011595;
B/B at genetic marker site GANE01015568;
B/B at genetic marker site WOUH023266.1;
B/B at genetic marker site WOUH023266.1.2;
A/A at genetic marker site WOUH01005557.1;
A/B at genetic marker site UDP74G1; and
B/B at genetic marker site UDP85C2.

15. The stevia plant of claim 1, wherein the plant comprises:
A/A at genetic marker site GANE01021955;
A/B at genetic marker site GANE01016707;
A/A at genetic marker site GANE01016455;
A/A at genetic marker site GANE01004406;
A/A at genetic marker site GANE01025251;
B/B at genetic marker site GANE01000877;
A/A at genetic marker site GANE01003004;
A/A at genetic marker site GANE01011595;
B/B at genetic marker site GANE01015568;
B/B at genetic marker site GANE01016432;
B/B at genetic marker site WOUH023266.1;
B/B at genetic marker site WOUH023266.1.2;
A/A at genetic marker site WOUH01005557.1;
A/B at genetic marker site UDP74G1; and
B/B at genetic marker site UDP85C2.

16. A plant, or a plant part thereof produced by growing the plant of claim 1, wherein the plant part is selected from the group consisting of leaves, cotyledons, hypocotyl, meristematic cells, ovules, seeds, cells, roots, root tips, pistils, anthers, flowers, and stems,

17. An extract of the stevia plant of claim 1, wherein the extract comprises one or more steviol glycosides.

18. A tissue or cell culture of regenerable cells produced from the plant of claim 1.

19. A method of producing stevia seeds, comprising:
(a) planting seeds of the stevia plant of claim 1;
(b) cultivating stevia plants resulting from the seeds until the plants bear flowers;
(c) allowing fertilization of the flowers of the plants; and
(d) harvesting seeds produced from the plants.

20. A method of vegetatively propagating a stevia plant, the method comprising:
a) collecting tissue or cells capable of being propagated from the plant according to claim 1;
b) cultivating the tissue or cells of (a) to obtain proliferated shoots; and
c) rooting said the shoots to obtain rooted plantlets or cultivating said tissue or cells to obtain proliferated shoots, or plantlets.


SEQUENCE LISTING (31 sequences)

SEQ ID NO: 1 - DNA, 20 bp, Artificial Sequence
  Synthetic primer
cggaacctcggaatcatctt

SEQ ID NO: 2 - DNA, 24 bp, Artificial Sequence
  Synthetic primer
aagaggaagaatgaagagaagagg

SEQ ID NO: 3 - DNA, 23 bp, Artificial Sequence
  Synthetic primer
ccttcgatagttctgacgcttac

SEQ ID NO: 4 - DNA, 22 bp, Artificial Sequence
  Synthetic primer
caatctcactaggaagggttcg

SEQ ID NO: 5 - DNA, 22 bp, Artificial Sequence
  Synthetic primer
gaaagctaccggagcagataaa

SEQ ID NO: 6 - DNA, 20 bp, Artificial Sequence
  Synthetic primer
tacatcggaatcagtcccga

SEQ ID NO: 7 - DNA, 23 bp, Artificial Sequence
  Synthetic primer
acaagtcatgatcagaccatctt

SEQ ID NO: 8 - DNA, 22 bp, Artificial Sequence
  Synthetic primer
cgttcttcctcctagcttcttc

SEQ ID NO: 9 - DNA, 23 bp, Artificial Sequence
  Synthetic primer
ccttcgatagttctgacgcttac

SEQ ID NO: 10 - DNA, 22 bp, Artificial Sequence
  Synthetic primer
caatctcactaggaagggttcg

SEQ ID NO: 11 - DNA, 20 bp, Artificial Sequence
  Synthetic primer
acccaagaacccgaatcagg

SEQ ID NO: 12 - DNA, 21 bp, Artificial Sequence
  Synthetic primer
agagacaaacctacccagtga

SEQ ID NO: 13 - DNA, 20 bp, Artificial Sequence
  Synthetic primer
acggtgaactgcgctgataa

SEQ ID NO: 14 - DNA, 20 bp, Artificial Sequence
  Synthetic primer
gatttgtggccgaggattgc

SEQ ID NO: 15 - DNA, 20 bp, Artificial Sequence
  Synthetic primer
attccgcactaccatacgcc

SEQ ID NO: 16 - DNA, 22 bp, Artificial Sequence
  Synthetic primer
tgaaagaccgaatgcaatacgc

SEQ ID NO: 17 - DNA, 20 bp, Artificial Sequence
  Synthetic primer
tcacgatcgtttggaaccgt

SEQ ID NO: 18 - DNA, 20 bp, Artificial Sequence
  Synthetic primer
ctgaccccatcttccccaac

SEQ ID NO: 19 - DNA, 20 bp, Artificial Sequence
  Synthetic primer
atgtcgctgtttcttcgggt

SEQ ID NO: 20 - DNA, 20 bp, Artificial Sequence
  Synthetic primer
ctttgtggggcttcagcaac

SEQ ID NO: 21 - DNA, 22 bp, Artificial Sequence
  Synthetic primer
tggaactacaatccctccaatc

SEQ ID NO: 22 - DNA, 22 bp, Artificial Sequence
  Synthetic primer
cgagtccttcgtagacgttaat

SEQ ID NO: 23 - DNA, 22 bp, Artificial Sequence
  Synthetic primer
tggaactacaatccctccaatc

SEQ ID NO: 24 - DNA, 22 bp, Artificial Sequence
  Synthetic primer
cgagtccttcgtagacgttaat

SEQ ID NO: 25 - DNA, 22 bp, Artificial Sequence
  Synthetic primer
cggaagtcctctagtgtttaag

SEQ ID NO: 26 - DNA, 21 bp, Artificial Sequence
  Synthetic primer
gattgaaagagccgaggtatc

SEQ ID NO: 27 - DNA, 20 bp, Artificial Sequence
  Synthetic primer
aatcgggccaacacttccat

SEQ ID NO: 28 - DNA, 24 bp, Artificial Sequence
  Synthetic primer
agtcgcaaaattttaggacaaaga

SEQ ID NO: 29 - DNA, 17 bp, Artificial Sequence
  Synthetic primer
agagagagagagagagc

SEQ ID NO: 30 - DNA, 20 bp, Artificial Sequence
  Synthetic primer
ccacattgtctagacggttc

SEQ ID NO: 31 - DNA, 21 bp, Artificial Sequence
  Synthetic primer
cggtgatttccttgaccattt